(19) United States
(12) Patent Application Publication
(10) Pub. No.: US 2012/0314046 A1
(43) Pub. Date: Dec. 13, 2012

(54) TIREDNESS STATE DETECTING SYSTEM AND METHOD

(75) Inventors: Yan ZHUANG, Shenzhen City (CN); Xiao-Jun FU, Shenzhen City (CN); Jin-Rong ZHAO, Shenzhen City (CN)

(73) Assignees: HON HAI PRECISION INDUSTRY CO., LTD, Tu-Cheng (TW); HONG FU JIN PRECISION INDUSTRY (ShenZhen) CO., LTD, Shenzhen City (CN)

(21) Appl. No.: 13/457,425

(22) Filed: Apr. 26, 2012

(30) Foreign Application Priority Data
Jun. 7, 2011 (CN) 201110150962.1

(51) Int. Cl. H04N 7/18; A61B 5/107

(52) U.S. Cl. 348/78; 600/595; 348/E07.085

(57) ABSTRACT

A computing device and method detects a tiredness state of a user. A camera positioned on a display device captures images of the user when the user is positioned in front of the camera. The computing device analyzes the images to obtain eye parameters of the eye of the user. The computing device reminds the user to have a rest, in response to a determination that the eye parameters of the eye of the user match predetermined eye parameters of the eye of the user.

### BACKGROUND

1. Technical Field

Embodiments of the present disclosure relate to detection technology, and particularly to a tiredness state detecting system and method.

2. Description of Related Art

A user may continuously use a personal computer (PC) for many purposes for many hours, such as, typing, coding, watching movies, chatting, or other things. However, staying in front of the PC may cause the user to be tired and influence a health of the user. Improved methods to detect when the user becomes tired are desirable.

### BRIEF DESCRIPTION OF THE DRAWINGS

FIG. 1 is a block diagram of one embodiment of a tiredness state detecting system.

FIG. 2 is a block diagram of one embodiment of a computing device of FIG. 1.

FIG. 3 is a flowchart of one embodiment of a tiredness state detecting method.

FIG. 4 illustrates one embodiment of an image of an eye of a user.

### DETAILED DESCRIPTION

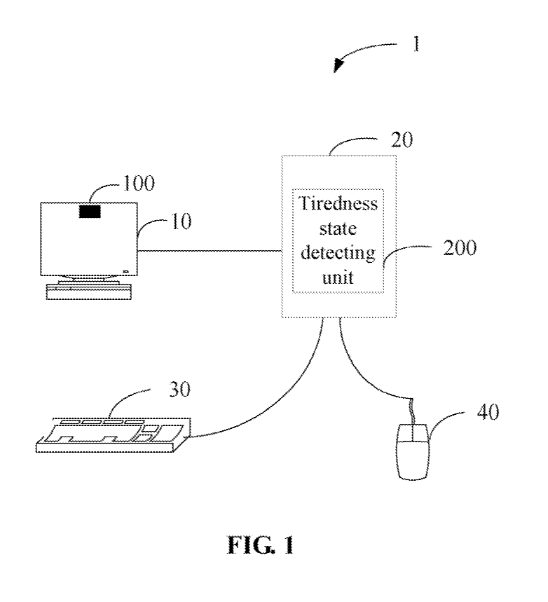

The disclosure is illustrated by way of example and not by way of limitation in the figures of the accompanying drawings in which like references indicate similar elements. It should be noted that references to “an” or “one” embodiment in this disclosure are not necessarily to the same embodiment, and such references mean at least one. The term “data” may refer to a single data item or may refer to a plurality of data items. These terms, with reference to FIGS. 1-4, will be described in greater detail below.

In general, the word “module”, as used herein, refers to logic embodied in hardware or firmware, or to a collection of software instructions, written in a programming language, such as, Java, C, or assembly. One or more software instructions in the modules may be embedded in firmware, such as in an EPROM. The modules described herein may be implemented as either software and/or hardware modules and may be stored in any type of non-transitory computing device-readable medium or other storage device. Some non-limiting examples of non-transitory computing device-readable media include CDs, DVDs, BLU-RAY, flash memory, and hard disk drives.

FIG. 1 is a block diagram of one embodiment of a tiredness state detecting system 1. The tiredness state detecting system 1 comprises a computing device 20, and a plurality of peripherals that are electronically connected to the computing device 20, such as a display device 10, a keyboard 30, and a mouse 40. The peripherals may be used to input or output various signals or interfaces. The display device 10 includes a camera 100, and the camera 100 may be positioned on a top position of the display device 10. The camera 100 captures images of a user that is positioned in front of the camera 100. Additionally, the computing device 20 may be electronically connected to a database system using open database connectivity (ODBC) or JAVA database connectivity (JDBC), for example. The database system may store the images which are captured by the camera 100 of the computing device 20. In one embodiment, the computing device 20 may be a personal computer (PC), a network server, or any other data-processing equipment.

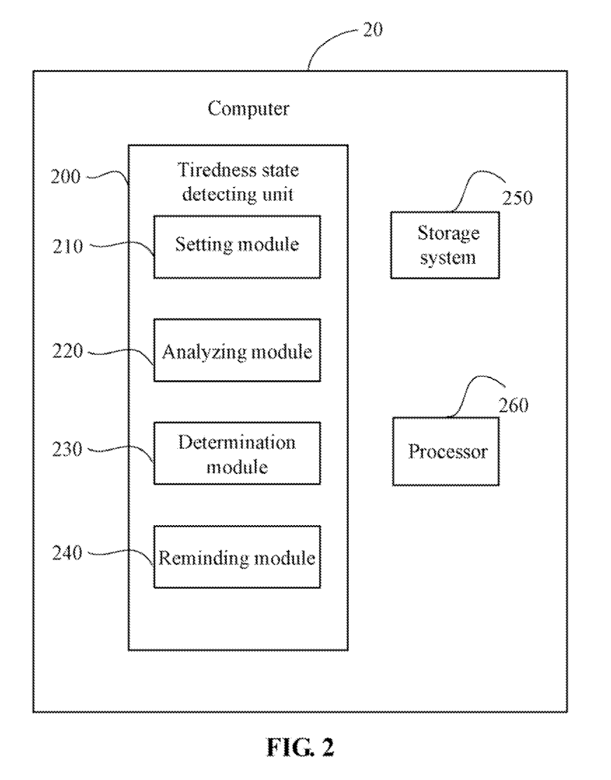

FIG. 2 is a block diagram of one embodiment of the computing device 20. The computing device 20 includes a tiredness state detecting unit 200. The tiredness state detecting unit 200 reminds a user to have a rest when the user is determined by the tiredness state detecting unit 200 to be tired. In one embodiment, the computing device 20 includes a storage system 250, and at least one processor 260. In one embodiment, the tiredness state detecting unit 200 includes a setting module 210, an analyzing module 220, a determination module 230, and a reminding module 240. The modules 210-240 may include computerized code in the form of one or more programs that are stored in the storage system 250. The computerized code includes instructions that are executed by the at least one processor 260 to provide functions for the modules 210-240. The storage system 250 may be a cache or a dedicated memory, such as an EPROM, HDD, or flash memory.

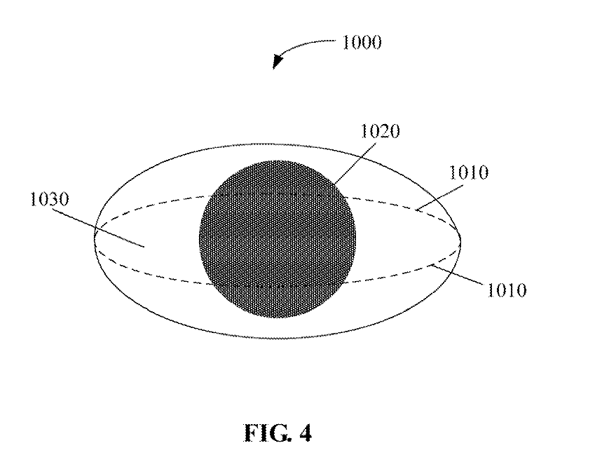

The setting module 210 sets predetermined eye parameters of an eye of a user when the user is not tired. The predetermined eye parameters include a percentage range of a white part 1030 of an eye 1000 (as shown in FIG. 4). In one embodiment, as shown in FIG. 4, the eye 1000 of the user includes eyelids 1010 (e.g., an upper eyelid and a lower eyelid), an iris 1020, and the white part 1030. The visible area of the white part 1030 may change according to a distance between the upper eyelid 1010 and the lower eyelid 1010. For example, if the eye 1000 is closed or nearly closed (e.g., the user is sleeping or tired), the upper eyelid 1010 is close to the lower eyelid 1010, and the upper eyelid 1010 and the lower eyelid 1010 cover more area of the white part 1030. When the user is not tired, then the eye 1000 of the user may be open, causing area of the white part 1030 may amount to 20%-25% of the total area of the eye 1000. When the user is tired, then the area of the white part 1030 may amount to less than 20% of the total area of the eye 1000.

The analyzing module 220 analyzes the images of the user and obtains eye parameters of the eye of the user from the images. In one embodiment, the eye parameters of the eye of the user include a percentage of the white part 1030 of the eye 1000. The analyzing module 220 can extract the eyes 1000 of the user in the image. For example, as shown in FIG. 4, the eye 1000 is extracted by the analyzing module 220 from an image. The analyzing module 220 calculates a number of the pixels of the eye 1000, and a number of the pixels of the white part 1030, and computes a percentage of the number of the pixels of the white part 1030 compared to the number of the pixels of the eye 1000. For example, if the eye 1000 includes five hundreds pixels, and the white part 1030 include one hundred pixels, the percentage of the white part 1030 of the eye 1000 is 20%.

The determination module 230 determines if the obtained eye parameters of the eye of the user match the predetermined eye parameters of the eye of the user. In one embodiment, if the percentage of the white part 1030 of eye 1000 falls within the percentage range of the white part 1030 of eye 1000 (e.g., 20%-25%), the eye parameters of the eye of the user is determined to match the predetermined eye parameters of the eye of the user. Otherwise, if the percentage of the white part 1030 of eye 1000 falls outside the percentage range of the white part 1030 of eye 1000 (e.g., 20%-25%), the eye parameters of the eye of the user is determined not to match the predetermined eye parameters of the eye of the user.

The reminding module 240 reminds the user to have a rest, in response to a determination that the obtained eye parameters of the eye of the user match the predetermined eye parameters of the eye of the user. In one embodiment, the reminding module 240 reminds the user using a speaker to output an audible announcement, such as, “Dear user, you are tired, please go outside and take a walk to relax”. The reminding module 240 may also remind the user by displaying a picture (e.g., a smiley face) on the display device 10. The user may feel relaxed when seeing the smiley face.

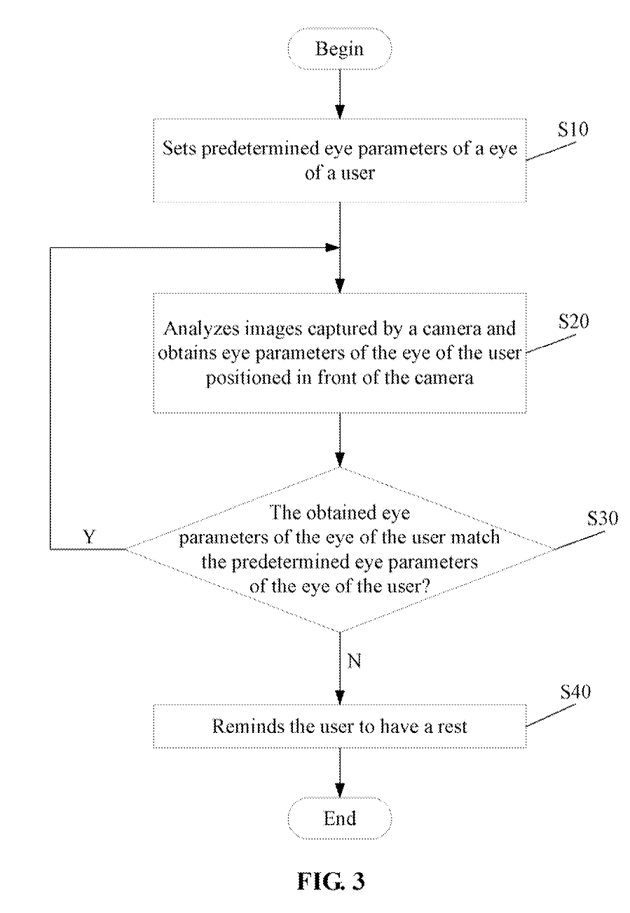

FIG. 3 is a flowchart of one embodiment of a tiredness state detecting method. Depending on the embodiment, additional steps may be added, others deleted, and the ordering of the steps may be changed.

In step S10, the setting module 210 sets predetermined eye parameters of an eye of a user. The predetermined eye parameters include a percentage range of a white part 1030 of an eye 1000. In one embodiment, the percentage range may be 20%-25%.

In step S20, the analyzing module 220 analyzes the images of the user and obtains eye parameters of the eye of the user from the images. As mentioned above, the eye parameters of the eye of the user include a percentage of a white part 1030 of an eye 1000. For example, if the eye 1000 includes five hundreds pixels, and the white part 1030 includes one hundred pixels, thus, the percentage of the white part 1030 of the eye 1000 is 20%.

In step S30, the determination module 230 determines if the obtained eye parameters of the eye of the user match the predetermined eye parameters of the eye of the user. In one embodiment, if the percentage of the white part 1030 of eye 1000 is 23%, the eye parameters of the eye of the user is determined to match the predetermined eye parameters of the eye of the user, the procedure returns to step S20. Otherwise, if the percentage of the white part 1030 of eye 1000 is 16%, the eye parameters of the eye of the user is determined not to match the predetermined eye parameters of the eye of the user, the procedure goes to step S40.

In step S40, the reminding module 240 outputs an indication to remind the user to have a rest. In one embodiment, the reminding module 240 uses a speaker of the computing device 20 to output the indication. The indication may be an audible announcement, such as, “Dear user, you are tired, please go outside and take a walk to relax”. The reminding module 240 may show a picture (smiley face) on the display device 10. The indication may be the picture. The user maybe feels relaxing when seeing the smiley face.

Although certain inventive embodiments of the present disclosure have been specifically described, the present disclosure is not to be construed as being limited thereto. Various changes or modifications may be made to the present disclosure without departing from the scope and spirit of the present disclosure.

What is claimed is:

1. A computing device, comprising:
a storage system;
at least one processor; and
one or more programs stored in the storage system and being executable by the at least one processor, the one or more programs comprising:
an analyzing module that obtains eye parameters of an eye of a user from images of the user positioned in front of a display device having a camera positioned on the display device;
a setting module that sets predetermined eye parameters of the eye of the user;
a determination module that determines if the obtained eye parameters of the eye of the user match the predetermined eye parameters of the eye of the user; and
a reminding module that outputs an indication to remind the user to have a rest, in response to a determination that the obtained eye parameters of the eye of the user match the predetermined eye parameters of the eye of the user.

2. The computing device of claim 1, wherein the predetermined eye parameters comprise a percentage range of a white part of the eye, and the eye parameters of the eye of the user comprise a percentage of the white part of the eye.

3. The computing device of claim 2, wherein the percentage range of the white part of the eye amounts to 20% to 25% area of the eye.

4. The computing device of claim 3, wherein the obtained eye parameters of the eye of the user match the predetermined eye parameters of the eye of the user upon the condition that the percentage of the white part of the eye falls within the percentage range of the white part of the eye.

5. The computing device of claim 1, wherein the indication is an audible announcement using a speaker of the computing device.

6. The computing device of claim 1, wherein the indication is a picture displaying on the display device.

7. A tiredness state detecting method implemented by a computing device, the method comprising:
obtaining eye parameters of an eye of a user from images of the user positioned in front of a display device having a camera positioned on the display device;
setting predetermined eye parameters of the eye of the user;
determining if the obtained eye parameters of the eye of the user match the predetermined eye parameters of the eye of the user; and
outputting an indication to remind the user to have a rest, in response to a determination that the obtained eye parameters of the eye of the user match the predetermined eye parameters of the eye of the user.

8. The method of claim 7, wherein the predetermined eye parameters comprise a percentage range of a white part of the eye, and the eye parameters of the eye of the user comprise a percentage of the white part of the eye.

9. The method of claim 8, wherein the percentage range of the white part of the eye amounts to 20% to 25% area of the eye.

10. The method of claim 9, wherein the obtained eye parameters of the eye of the user match the predetermined eye parameters of the eye of the user upon the condition that the percentage of the white part of the eye falls within the percentage range of the white part of the eye.

11. The method of claim 7, wherein the indication is an audible announcement using a speaker of the computing device.

12. The method of claim 7, wherein the indication is a picture displaying on the display device.

13. A non-transitory computing device-readable medium having stored thereon instructions that, when executed by a computing device, causing the computing device to perform a tiredness state detecting method, the method comprising:
obtaining eye parameters of an eye of a user from images of the user positioned in front of a display device having a camera positioned on the display device;
setting predetermined eye parameters of the eye of the user;
determining if the obtained eye parameters of the eye of the user match the predetermined eye parameters of the eye of the user; and
outputting an indication to remind the user to have a rest, in response to a determination that the obtained eye parameters of the eye of the user match the predetermined eye parameters of the eye of the user.

14. The non-transitory medium of claim 13, wherein the predetermined eye parameters comprise a percentage range of a white part of the eye, and the eye parameters of the eye of the user comprise a percentage of the white part of the eye.

15. The non-transitory medium of claim 14, wherein the percentage range of the white part of the eye amounts to 20% to 25% area of the eye.

16. The non-transitory medium of claim 15, wherein the obtained eye parameters of the eye of the user match the predetermined eye parameters of the eye of the user upon the condition that the percentage of the white part of the eye falls within the percentage range of the white part of the eye.

17. The non-transitory medium of claim 13, wherein the indication is an audible announcement using a speaker of the computing device.

18. The non-transitory medium of claim 13, wherein the indication is a picture displaying on the display device.
